(19) United States
(12) Patent Application Publication
(10) Pub. No.: US 2005/0049268 A1
(43) Pub. Date: Mar. 3, 2005

(54) Use of at least one abl, kit and/or platelet-derived growth factor receptor tyrosine kinases inhibitor for treating hair depigmentation

(76) Inventors: Francois-Xavier Mahon, Bordeaux (FR); Pascale Cony-Makhoul, Annecy (FR); Gabriel Etienne, Bordeaux (FR)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 10/496,055

(22) PCT Filed: Nov. 20, 2002
(86) PCT No.: PCT/EP02/13021

(30) Foreign Application Priority Data
Nov. 21, 2001 (GB) 0127922.3

(51) Int. Cl. A61K 31/506

(52) U.S. Cl. 514/275

(57) ABSTRACT

The invention relates to a method of treating a warm-blooded animal, especially a human, having depigmented hair or subject to hair depigmentation comprising administering to the animal a useful amount of a) at least one Abl, Kit and/or platelet-derived growth factor (PDGF) receptor tyrosine kinases inhibitor, the use of a) and at least one cosmetic acceptable carrier, to retard, prevent, suppress, and/or revere the depigmentation of hair, the use of a) for the preparation of a medicament for the treatment of a disease characterized by hair depigmentation and a topical cosmetic composition comprising a useful amount of at least one Abl, Kit or PDGF receptor tyrosine kinases inhibitor.

The invention relates to a method of treating a warm-blooded animal, especially a human, having depigmented hair or subject to hair depigmentation comprising administering to the animal a useful amount of a) at least one Abl, Kit and/or platelet-derived growth factor (PDGF) receptor tyrosine kinases inhibitor, especially the N-{5-[4-(4-methyl-piperazino-methyl)-benzoylamido]-2-methylphenyl}-4-(3-pyridyl)-2-pyrimidine-amine or a pharmaceutically acceptable salt thereof, the use of a) and at least one cosmetic acceptable carrier, to retard, prevent, suppress, and/or reverse the depigmentation of hair; the use of a) for the preparation of a medicament for the treatment of a disease characterized by hair depigmentation and a topical cosmetic composition comprising a useful amount of at least one Abl, Kit or PDGF receptor tyrosine kinases inhibitor especially N-{5-[4-(4-methyl-piperazino-methyl)-benzoylamido]-2-methylphenyl}-4-(3-pyridyl)-2-pyrimidine-amine or a pharmaceutically acceptable salt thereof.

Abelson tyrosine kinase (i.e. Abl, c-Abl) is involved in the regulation of the cell cycle, in the cellular response to genotoxic stress, and in the transmission of information about the cellular environment through integrin signaling. Overall, it appears that the Abl protein serves a complex role as a cellular module that integrates signals from various extracellular and intracellular sources and that influences decisions in regard to cell cycle and apoptosis. Abelson tyrosine kinase includes also sub-types derivatives such as the chimeric fusion protein (oncoprotein) BCR-Abl with deregulated tyrosine kinase activity or the v-Abl. BCR-Abl, is critical in the pathogenesis of 95% of chronic myelogenous leukemia (CML) and 10% of acute lymphocytic leukemia.

The receptor tyrosine kinase (Kit) is expressed by and critical for the development and growth of mast cells, melanocytes, hematopoetic stem cells, and the interstitial cells of Cajal. Kit protein that is not bound to SCF exists predominantly as an enzymatically inactive monomer spanning the cell plasma membrane. SCF exists predominantly as a bivalent dimer, and can be expressed on the surface of stromal cells or released as a soluble molecule by protease cleavage. SCF binding induces Kit dimerization and auto-phosphorylation on tyrosines in the intracellular domain of the receptor. This auto-phosphorylation of specific tyrosine residues creates docking sites for signal transduction molecules and induces substrate binding and phosphorylation. Mutations which cause ligand independent constitutive phosphorylation and activation of Kit have been shown to transform cell lines from factor-dependent growth to factor-independent growth in vitro, and can transform indolent tumors to aggressive tumors in vivo. The receptor tyrosine kinase (Kit) also comprises sub-types derivatives such as the chimeric fusion proteins (oncoprotein) c-Kit or v-Kit, with deregulated tyrosine kinase activity.

Platelet-derived growth factor (PDGF) receptor tyrosine kinase is a very commonly occurring growth factor, which plays an important role both in normal growth and also in pathological cell proliferation, such as is seen in carcinogenesis and in diseases of the smooth-muscle cells of blood vessels, for example in atherosclerosis and thrombosis. The inhibition of PDGF-stimulated receptor tyrosine kinase activity (inhibition of receptor phosphorylation) in vitro can be measured in PDGF receptor immune complexes of BALB/c 3T3 cells, as described by E. Andrejauskas-Buchdunger and U. Regenass in Cancer Research 52, 5353-5358 (1992). Inhibitors of PDGF receptor tyrosine kinase activity are well known in the art.

A number of compounds are known to inhibit the proliferation of cells by way of inhibition of either the abl-, the PDGF-Receptor and/or the Kit receptor tyrosine kinase. For example, International Application No. WO 97/02266, International Patent Application WO98135958 and especially European Patent Application EP 0 564 409-A, as well as International Application No. WO99/03854, all of which are incorporated by reference herewith, mention compounds that are inhibitors of at least one of the tyrosine kinases mentioned above. The preferred compound described in the above-mentioned patent applications is N-{5-[4-(4-methyl-piperazino-methyl)-benzoylamido]-2-methylphenyl}-4-(3-pyridyl)-2-pyrimidine-amine.

The preparation of N-{5-[4-(4-methyl-piperazino-methyl)-benzoylamido]-2-methylphenyl}-4-(3-pyridyl)-2-pyrimidine-amine (hereinafter: “Imatinib” [International Non-proprietary Name]) and the use thereof, especially as an antiproliferative agent, are described in EP-A-0 564 409, which was published on 6 Oct. 1993, in U.S. Pat. No. 5,521,184 issued May 28, 1994 or in JP 2 706 682.

It will be understood that references to Imatinib meant to also include the pharmaceutically acceptable salts. Imatinib or a pharmaceutically acceptable salt thereof may also be used in form of a hydrate or include other solvents used for crystallization. N-{5-[4-(4-methyl-piperazino-methyl)-benzoylamido]-2-methylphenyl}-4-(3-pyridyl)-2-pyrimidine-amine, is preferably used in the present invention in the form of its monomesylate salt.

The tyrosine kinase inhibitor Imatinib has recently shown promising results in the treatment of chronic myelogenous leukaemia (CML) and gastro-intestinal stroma tumors (GIST). Imatinib is a protein-tyrosine kinase inhibitor that is currently in clinical trials for the treatment of chronic myelogenous leukemia. Imatinib selectively inhibits the Abl and platelet-derived growth factor (PDGF) receptor tyrosine kinases in vitro and blocks cellular proliferation and tumor growth of Bcr-abl or v-abl-expressing cells. Imatinib was further found to potently inhibit the kinase activity of the α- and β-PDGF receptors and the receptor for stem cell factor, but not the closely related c-Fms, Flt-3, Kdr, Flt-1, and Tek tyrosine kinases. Additionally, no inhibition of c-Met or nonreceptor tyrosine kinases such as Src and Jak-2 has been observed. In cell-based assays, Imatinib selectively inhibited PDGF and stem cell factor-mediated cellular signaling, including ligand-stimulated receptor autophosphorylation, inositol phosphate formation, and mitogen-activated protein kinase activation and proliferation. These results expand the profile of Imatinib and suggest that in addition to chronic myelogenous leukemia, Imatinib may have clinical potential in the treatment of diseases that involve abnormal activation of Kit (i.e. c-Kit), Abl or PDGF receptor tyrosine kinases.

Pursuing clinical trials, the applicant has surprisingly discovered that Imatinib is useful for the pigment restoration in human subjects. This compound shows an unexpected high potency to retard, prevent, suppress, and/or even reverse the graying of hair. Thus, at least for some already-turned gray individuals, the natural hair color can be restored, and maintained, naturally without the use of dyes, colorants, or the like. And, at least for some not-yet turned gray individuals, the natural hair color can be maintained for longer periods (and perhaps indefinitely) than would have otherwise been the case.

The present invention thus concerns the use of at least one Abl, Kit and/or PDGF receptor tyrosine kinases inhibitor and at least one cosmetic acceptable carrier, to retard, prevent, suppress, and/or reverse the depigmentation of hair.

Preferably, the Abl, Kit and/or PDGF receptor tyrosine kinases inhibitor is N-{5-[4-(4-methyl-piperazino-methyl)-benzoylamido]-2-methylphenyl}-4-(3-pyridyl)-2-pyrimidine-amine or a pharmaceutically acceptable salt thereof.

The present invention more particularly concerns the use of at least one Abl, Kit and/or PDGF receptor tyrosine kinases inhibitor for the preparation of a medicament for the treatment of a a disease characterized by hair depigmentation.

Preferably, the Abl, Kit and/or PDGF receptor tyrosine kinases inhibitor used for the preparation of a medicament is N-{5-[4-(4-methyl-piperazino-methyl)-benzoylamido]-2-methylphenyl}-4-(3-pyridyl)-2-pyrimidine-amine or a pharmaceutically acceptable salt thereof.

In still another embodiment, the instant invention provides a method of treating a warm-blooded animal, especially a human, having depigmented hair or subject to hair depigmentation comprising administering to the animal a useful amount of at least one Abl, Kit or PDGF receptor tyrosine kinases inhibitor.

Preferably, the Abl, Kit and/or PDGF receptor tyrosine kinases inhibitor used in this method is N-{5-[4-(4-methyl-piperazino-methyl)-benzoylamido]-2-methylphenyl}-4-(3-pyridyl)-2-pyrimidine-amine or a pharmaceutically acceptable salt thereof.

In another embodiment, the instant invention relates to a topical cosmetic composition comprising a useful amount of at least one Abl, Kit and/or PDGF receptor tyrosine kinases inhibitor.

Preferably, the Abl, Kit and/or PDGF receptor tyrosine kinases inhibitor used in the composition is N-{5-[4-(4-methyl-piperazino-methyl)-benzoylamido]-2-methylphenyl}-4-(3-pyridyl)-2-pyrimidine-amine or a pharmaceutically acceptable salt thereof.

By the term “abl-, PDGF-R- and/or Kit receptor-tyrosine kinase inhibitor” preferably one of the following compounds is meant:

A compound mentioned in International Application No. WO 97/02266, International Patent Application WO98135958 and especially European Patent Application EP 0 564 409-A, as well as International Application No. WO99/03854, all of which are incorporated by reference herewith; Tyrphostin AG957 (see Kaur et al., Anticancer Drugs 5, 213-222 (1994), Herbimycin A (Okabe and Uehara, Leukemia and Lymphoma 12, 2156-2162 (1994), Blood 80, 1330-1338 (1994) and Leuk. Res. 18, 213-220 (1994), as well as Rioran et al., Oncogene 16, 133-1542 (1998)); Tyrphostins AG 1295, AG 1296 (see Kovalenko et al., Cancer Res. 54, 6106-6114 (1994), Lipson et al., Pharmacol & Exp- Therap. 285, 844-852 (1998), Krystal et al., Cancer Res. 57, 2203-2208 (1997)); SU 101 (Leflunomide), as well as its metabolite (see Shawer et al., Clin. Cancer Res. 3, 1167-1177 (1997), Mattar et al., FEBS Lett. 334, 161-164 (1993), Cherwinskyi et al., Inflamm. Res. 3, 1167-1177 (1997), and Strawn et al., Exp. Opin. Invest. Drugs 7, 533-573 (1998)); and Pyridopyrimidines (see e.g. Hamby et al., J. Med. Chem. 40, 2296-2303 (1997), Dahring et al., J. Pharmacol. Exp. Ther. 281, 1446-1456 (1997), Klutcho et al., Life Sci. 62, 143-150 (1998), Panek et al., J. Pharmacol. Exp. Ther. 283, 1433-1444 (1997), Boschelli et al., J. Med. Chem. 41, 4365-4377 (1998)). All the references mentioned above are incorporated herein by reference. The tyrosine kinase inhibitors, their synthesis and their use can be deduced from these references.

The term “and/or” used in “abl-, PDGF-R- and/or Kit receptor-tyrosine kinase inhibitor” means that either one or more of the mentioned tyrosine kinases is inhibited by a compound encompassed by this expression.

Gray hair, is often considered to be inevitable part of life and the aging process. Graying of hair generally results from a gradual replacement of pigmented hair by unpigmented hair as the melanocytes shut down pigment production as one gets older. This graying process often starts at around age forty (although it can begin much earlier or later) with the onset and rate of graying apparently controlled mainly by genetics. By some estimates, approximately 50 percent of all women will be at least partially gray by the age of fifty. In most cases, the graying process has generally been considered irreversible; once the hair follicle starts to produce gray hair, it is not likely to change back. Thus, for most individuals with graying or already-turned gray hair the options are limited: acceptance of the situation or masking with colorants, bleaches, dyes, highlights, head coverings, or wigs. Once coloring techniques are used, however, they must be repeated (or at least touched up) on a regular basis to maintain the color and avoid undesirable gray roots.

Hair depigmentation can also be the consequence of several diseases or therapeutic treatments.

It would be desirable, therefore, to provide alternatives for combating gray hair, especially ones which will increase and promote the overall healthiness of the hair. It would also be desirable to provide a method by which, at least in some cases, the onset of gray hair can be significantly delayed or even prevented. It would also be desirable to provide a method by which, at least in some cases, already-turned gray hair can be restored to its original natural color. The present invention provides such benefits and advantages.

By “disease characterized by hair depigmentation” the applicant means the treatment of a diseases involving a hair pigmentation disorder such as but not limited to Vitiligo or Piebaldism.

By “depigmentation” the applicant means loss of natural color. The loss of natural color can result in the graying of hair, which may be due to aging process, stress or diseases.

Vitiligo (vit-ill-EYE-go) is a pigmentation disorder in which melanocytes (the cells that make pigment) in the skin, the mucous membranes (tissues that line the inside of the mouth and nose and genital and rectal areas), and the retina (inner layer of the eyeball) are destroyed. As a result, white patches of skin appear on different parts of the body. The hair that grows in areas affected by vitiligo usually turns white because of depigmentation.

Piebaldism is an autosomal dominant disorder of melanocyte development characterized by white skin (leukoderma) and white hair (poliosis). In general, piebaldism has been distinguished from vitiligo by the presence of lesions from birth, the hyperpigmented macules of depigmented and normal skin, and the static course.

In the present description, the term “treatment” includes both prophylactic or preventative treatment as well as curative or disease suppressive treatment, including treatment of patients at risk of contracting depigmentation as well as ill patients. This term further includes the treatment for the delay of progression of the disease.

By “suppress and/or reverse the depigmentation” the applicant means that the natural hair color can be restored and/or maintained.

The term “curative” or “prevent” as used herein mean efficacy in treating ongoing episodes of depigmentation. The natural hair color can be restored and/or maintained.

The term “prophylactic” or “prevent” mean the prevention of the onset or recurrence of hair depigmentation.

The term “delay of progression” as used herein means administration of the active compound to patients being in a pre-stage or in an early phase of depigmentation, in which patients for example the natural hair color can be maintained for longer periods (and perhaps indefinitely).

To demonstrate that Abl, Kit and/or platelet-derived growth factor (PDGF) receptor tyrosine kinases inhibitors are suitable to retard, prevent, suppress, and/or even reverse the graying of hair clinical trials can be carried out in a manner known to the skilled person.

In the applicant clinical study 133 patients with a confirmed diagnosis of Philadelphia chromosome-positive CML received treatment with orally administered Imatinib (400 or 600 mg/j) according to the Novartis protocols. Nine of them (6.7%) with initial grey hair have presented progressive hair repigmentation (head in 8 cases, body and head in one case) under Imatinib treatment. Change in hair pigmentation occurred after a median treatment time of 5 months (range 2 to 14). In most of the cases, the phenomenon became apparent after 3 to 6 months of treatment.

The precise dosage of Abl, Kit and/or PDGF receptor tyrosine kinases inhibitor to be employed to retard, prevent, suppress, and/or even reverse the graying of hair depends upon several factors including the host, the nature and the severity of the condition being treated, the mode of administration.

However, in general, satisfactory effect is achieved when the Abl, Kit and/or PDGF receptor tyrosine kinases inhibitor is administered parenterally, e.g., intraperitoneally, intravenously, intramuscularly, subcutaneously, rectally, topically or enterally.

Imatinib can be prepared and administered as described in WO 99/03854, especially the monomesylate salt of N-{5-[4-(4-methyl-piperazino-methyl)-benzoylamido]-2-methylphenyl}-4-(3-pyridyl)-2-pyrimidine-amine can be formulated as described in examples 4 and 6 of WO 99/03854.

N-{5-[4-(4-methyl-piperazino-methyl)-benzoylamido]-2-methylphenyl}-4-(3-pyridyl)-2-pyrimidine-amine or a pharmaceutically acceptable salt thereof, is preferably administered to a human in a dosage in the range of about 2.5 to 850 mg/day, more preferably 5 to 600 mg/day and most preferably 20 to 300 mg/day. Unless stated otherwise herein, the compound is preferably administered from one to four times per day, more preferably once daily.

Usually, a small dose is administered initially and the dosage is gradually increased until the optimal dosage for the host under treatment is determined. The upper limit of dosage is that imposed by side effects and can be determined by trial for the host being treated.

The Abl, Kit and/or PDGF receptor tyrosine kinases inhibitor may be combined with one or more pharmaceutically acceptable carriers and, optionally, one or more other conventional pharmaceutical adjuvants and administered topically, enterally, e.g. orally, in the form of tablets, capsules, caplets, etc. or parenterally, e.g., intraperitoneally or intravenously, in the form of sterile injectable solutions or suspensions. The enteral and parenteral compositions may be prepared by conventional means.

The infusion solutions according to the present invention are preferably sterile. This may be readily accomplished, e.g. by filtration through sterile filtration membranes. Aseptic formation of any composition in liquid form, the aseptic filling of vials and/or combining a pharmaceutical composition of the present invention with a suitable diluent under aseptic conditions are well known to the skilled addressee.

The Abl, Kit and/or PDGF receptor tyrosine kinases inhibitor may be formulated into topical, enteral and parenteral pharmaceutical compositions containing an amount of the active substance that is effective for inhibiting tyrosine kinase activity, such compositions in unit dosage form and such compositions comprising a pharmaceutically acceptable carrier. Examples of useful compositions are described in examples 4 and 6 of WO 99/03854.

Preferably the Abl, Kit and/or PDGF receptor tyrosine kinases inhibitor is formulated into topical pharmaceutical composition. Most preferably such topical pharmaceutical composition is a pharmaceutical composition for transdermal administration as described hereafter.

In a further embodiment, the Abl, Kit and/or PDGF receptor tyrosine kinases inhibitor is administered by a cosmetic composition for topical application in order to prevent hair from turning grey and promoting natural hair repigmentation, while reducing and/or eliminating any negative side-effect. As used in this application, “negative side effects” refers to side effects, which are harmful or undesirable to the subject of the treatment.

It will also be understood that cosmetic carriers can be formulated in various forms such as an ointment, hair cream, hair tonic, hair lotion, hair mousse, hair gel, hair spray, hair shampoo, hair rinse and so forth. The carrier can be in a wide variety of forms. The topical skin composition of the present invention may appropriately contain a variety of ingredients or carriers, which are usually employed in cosmetics. Examples of such ingredients or carriers include humectants, powders, gelation agents, thickeners, surfactants, emulsifiers, anti-inflammatory agents, antioxidants, pH regulating agents, chelating agents, preservatives, dyes, perfumes, existing skin-aging preventive or retarding agents such as collagen. The composition may be produced through a conventional method in accordance with the application form. For example, emulsion carriers, including, but not limited to, oil-in-water, water-in-oil, water-in-oil-in-water, and oil-in-water-in-silicone emulsions (e.g., silicone-in-water or water-in-silicone), are useful herein.

Emulsifiers may be nonionic, anionic or cationic. Suitable emulsifiers are disclosed in, for example, U.S. Pat. No. 3,755,560, issued Aug. 28, 1973; U.S. Pat. No. 4,421,769, issued Dec. 20, 1983; and McCutcheon's Detergents and Emulsifiers, North American Edition, pages 317-324 (1986).

The emulsion may also contain an anti-foaming agent to minimize foaming upon application to the skin. Anti-foaming agents include high molecular weight silicones and other materials well known in the art for such use.

The term “topical application”, as used herein, means to apply or spread the compositions of the present invention onto the surface of mammalian keratinous tissue.

The term “keratinous tissue,” as used herein, refers to keratin-containing layers disposed as the outermost protective covering of mammals, which includes, but is not limited to, skin, hair.

The term “cosmetic acceptable carrier” as used herein, means that the compositions or components thereof so described are suitable for use in contact with human keratinous tissue without undue toxicity, incompatibility, instability, allergic response, and the like.

Cosmetic Methods (or cosmetic uses) of applying the herein described hair repigmentation promoters are limited only in that they should be allowed to retard, prevent, suppress, and/or reverse the depigmentation of hair and act as a cosmetic.

Typically, the hair repigmentation promoter is applied in a water carrier as a spray directly to the treatment area. It is believed that optimal results are achieved when the hair treatment composition is applied directly to the treatment area, in discrete applications, twice to three times a day in an amount sufficient to wet the skin or scalp. While some benefit may be noticed in a few moths, the treatment should be carried out for at least about 3 to 6 months.

It can be demonstrated experimentally that the new topical cosmetic composition is capable of enabling three important results to be achieved, ie. preventing hair from turning gray, and promoting natural hair repigmentation with limited undesirable side-effects.

Preferably the topical cosmetic and/or pharmaceutical composition is capable of transdermal penetration.

The transdermal administration of active principles represents a persuasive technique since it is non-invasive (i.e. local action) and is endowed with certain advantages such as the absence of gastrointestinal and other side effects or of degradation of the active substance by liver enzymes. When an active compound is transported through the skin, the horny layer of skin acts as a barrier against the permeation of the compound. Accordingly, various absorption promoters, or permeation enhancers, which facilitate the drug permeation through the horny layer, have been used in transdermal compositions such as teached in U.S. 2001023261 published on Sep. 20, 2001. The skin penetration enhancer reduces the diffusional resistance of the skin and promotes the distribution of the compound into the lipophilic part of the skin by modifying the physicochemical properties of the keratotic layer. The skin penetration enhancers which may be used in the present invention include a fatty acid and a derivative thereof, a fatty alcohol and a derivative thereof, an amide, a terpene, a sulfactant and a mixture thereof.

### EXPERIMENTAL PART

In our clinical study 9 patients with a confirmed diagnosis of Philadelphia chromosome-positive CML (chronic myelogenous leukemia) received treatment with orally administered Imatinib (400 or 600 mg/j). These patients have presented progressive hair repigmentation (head in 8 cases, body and head in one case) under Imatinib treatment. All patients (median age 63.4 years; range, 53 to 75; males=5, females=4) had Philadelphia chromosome-positive CML (6 chronic phase, 3 accelerated phase). Six of the 9 patients received Imatinib 400 mg/day and 3 received 600 mg/day. Median follow-up was 8.5 months (range 4 to 16). Previous therapies for CML included interferon-α (IFN-α) in all patients and autologous stem cell transplantation (n=4), hydroxyurea (n=3), cytosine arabinoside (n=1) which was discontinued at least 15 days before Imatinib. The median time between stopping IFN-α and starting Imatinib was 5.7 months (range 0.5 to 42). All other medicines unrelated to CML were maintained without modification. Change in hair pigmentation occurred after a median treatment time of 5 months (range 2 to 14). In most of the cases, the phenomenon became apparent after 3 to 6 months of treatment. In all 9 patients, the change in hair pigmentation may be considered to be an unexpected Imatinib-related effect.

1. Use of at least one Abl, Kit and/or PDGF receptor tyrosine kinases inhibitor and at least one cosmetic acceptable carrier, to retard, prevent, suppress, and/or reverse the depigmentation of hair.

2. Use according to claim 1, wherein the Abl, Kit and/or PDGF receptor tyrosine kinases inhibitor is N-{5-[4-(4-methyl-piperazino-methyl)-benzoylamido]-2-methylphenyl}-4-(3-pyridyl)-2-pyrimidine-amine or a pharmaceutically acceptable salt thereof.

3. Use of at least one Abl, Kit and/or PDGF receptor tyrosine kinases inhibitor for the preparation of a pharmaceutical composition for the treatment a disease characterized by hair depigmentation.

4. Use according to claim 3, wherein the Abl, Kit and/or PDGF receptor tyrosine kinases inhibitor is N-{5-[4-(4-methyl-piperazino-methyl)-benzoylamido]-2-methylphenyl}-4-(3-pyridyl)-2-pyrimidine-amine or a pharmaceutically acceptable salt thereof.

5. Use according to claim 4, wherein the disease characterized by hair depigmentation is vitiligo or piebaldism.

6. A method of treating a warm-blooded animal, especially a human, having depigmented hair or subject to hair depigmentation comprising administering to the animal a useful amount of at least one Abl, Kit or PDGF receptor tyrosine kinases inhibitor.

7. A method according to claim 6, wherein the Abl, Kit and/or PDGF receptor tyrosine kinases inhibitor is N-{5-[4-(4-methyl-piperazino-methyl)-benzoylamido]-2-methylphenyl}-4-(3-pyridyl)-2-pyrimidine-amine or a pharmaceutically acceptable salt thereof.

8. A topical cosmetic composition comprising a useful amount of at least one Abl, Kit and/or PDGF receptor tyrosine kinases inhibitor and a cosmetic acceptable carrier.

9. A topical cosmetic composition according to claim 8, wherein the Abl, Kit and/or PDGF receptor tyrosine kinases inhibitor is N-{5-[4-(4-methyl-piperazino-methyl)-benzoylamido]-2-methylphenyl}-4-(3-pyridyl)-2-pyrimidine-amine or a pharmaceutically acceptable salt thereof.
